(19) United States
(12) Patent Application Publication
(10) Pub. No.: US 2009/0215849 A1
(43) Pub. Date: Aug. 27, 2009

(54) High stability aqueous gels comprising metronidazole and methods for the preparation thereof

(75) Inventors: Leila Zarif, Peymeinade (FR); Gerald Pedrassi, Draguignan (FR); Alain Brzokewicz, Antibes (FR)

Correspondence Address: BUCHANAN, INGERSOLL & ROONEY PC, POST OFFICE BOX 1404, ALEXANDRIA, VA, 22313-1404, US

(73) Assignee: GALDERMA S.A., CHAM (CH)

(21) Appl. No.: 11/892,292

(22) Filed: Aug. 21, 2007

Related U.S. Application Data
(63) Continuation of application No. PCT/EP2006/002074, filed on Feb. 16, 2006.
(60) Provisional application No. 60/671,635, filed on Apr. 15, 2005.

(30) Foreign Application Priority Data
Feb. 25, 2005 (FR) 0501949

(51) Int. Cl. A61K 31/4164; A61P 17/00

(52) U.S. Cl. 514/398

(57) ABSTRACT

A method for preparing highly stable aqueous gels based on metronidazole, well suited for treating such skin conditions as rosacea, acne and dermatitis, entails the successive steps of:

(A) forming a solvent medium M comprising water and propylene glycol;

(B) dissolving the metronidazole in this solvent medium M, and optionally diluting the medium obtained by addition of water, whereby a solution S of metronidazole is obtained; and thence

(C) mixing the solution S obtained with a gelling polymer, in a sufficient amount to ensure gelling of the composition.

### CROSS-REFERENCE TO PRIORITY/PCT/PROVISIONAL APPLICATIONS

This application claims priority under 35 U.S.C. § 119 of FR 05/01949, filed Feb. 25, 2005, and of Provisional Application No. 60/671,635, filed Apr. 15, 2005, and is a continuation of PCT/EP 2006/002074 filed Feb. 16, 2006 and designating the United States, published in the English language as WO 2006/089804 A1 on Aug. 31, 2006, each hereby expressly incorporated by reference in its entirety and each assigned to the assignee hereof.

### BACKGROUND OF THE INVENTION

1. Technical Field of the Invention

The present invention relates to a novel method for preparing metronidazole-based aqueous gels which are useful, in particular, as topical dermatological compositions, especially for the treatment of dermatoses, such as rosacea.

2. Description of Background and/or Related and/or Prior Art

Metronidazole, or 1-(2-hydroxyethyl)-2-methyl-5-nitroimidazole, is the compound having the formula (I):

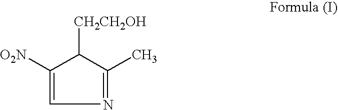

This compound and process for the preparation thereof are well known and are described in particular in U.S. Pat. No. 2,944,061.

Metronidazole is an acknowledged anti-bacterial and anti-parasitic agent useful for the treatment of a variety of conditions. This compound is known in particular as being particularly effective in the treatment of skin disorders such as rosacea.

Rosacea is a chronic skin condition which affects mainly adults. It is a type of dermatosis with recurrent symptoms, including in particular erythemas, papules, pustules, rhinophymas and/or telangiectasias, which manifests itself mainly in the region of the nose, the cheeks and the forehead.

For the treatment of such conditions, metronidazole is preferably administered by the topical route. Indeed, administration by the systemic route, in particular by the oral route, leads, in most cases, to undesirable side effects, such as gastrointestinal intolerance or vaginitis, to which other chronic disorders may also be added in the case of a long-term regimen.

Various topical formulations have been proposed for the topical administration of metronidazole, which are mainly oil-based compositions, in particular creams (oil-in-water emulsions) or ointments (in particular compositions based on petroleum jelly). In these compositions, metronidazole is dissolved in the oily phase. These oil-based compositions have the advantage of being able to contain large quantities of metronidazole in the solubilized state, available for topical application. However, they are found, in practice, to be poorly suited to dermatological use. Indeed, they require the presence of ingredients, in particular oils, emulsifiers or surfactants, which are found to exhibit comedogenic, acneigenic, drying and/or irritating properties for the skin. Furthermore, patients treated with compositions of this type often feel sensations of burning or urtication.

To avoid these problems, there have been proposed, in U.S. Pat. No. 4,837,378, compositions in the form of aqueous gels which do not require the presence of comedogenic, acneigenic, drying and/or irritating agents present in the abovementioned oil-based compositions.

The compositions of U.S. Pat. No. 4,837,378 comprise metronidazole in an aqueous phase gelled with a polymeric gelling agent, such as a polycarboxylated vinyl polymer. The method for preparing these compositions, as described in U.S. Pat. No. 4,837,378, entails mixing the gelling polymer with an aqueous solution of metronidazole.

The compositions described in U.S. Pat. No. 4,837,378 may additionally comprise additional agents, such as propylene glycol which improve the efficacy of administration of metronidazole, or preservatives such as methylparaben or propylparaben. Where appropriate, these additives are added to the preformed solution of metronidazole in water, and then the mixture obtained is mixed with the gelling polymer.

The aqueous gels of U.S. Pat. No. 4,837,378 have numerous advantages, in addition to the fact that they do not comprise ingredients leading to the side effects observed with the abovementioned oil-based compositions. In particular, they allow better control of application and a uniform distribution of the active ingredient. Furthermore, they act as prolonged-release systems, which gradually deliver the active ingredient topically, in a therapeutically effective amount for a prolonged period.

### SUMMARY OF THE INVENTION

The present invention provides aqueous gels for the topical administration of metronidazole having the advantages of the compositions of U.S. Pat. No. 4,837,378, but additionally having increased stability over time.

For the purposes of the present description, the expression “stability” of an aqueous metronidazole gel means its capacity to retain its gel structure over a long period, with metronidazole mainly in the dissolved and non-crystallized state. It is considered that a gel is all the more stable if the period during which is retains its gel structure with metronidazole mainly in the dissolved state is lengthy.

To this end, according to a first aspect, the present invention features a method for preparing a stable aqueous gel based on metronidazole, comprising the successive steps:

(A) forming a solvent medium M comprising water and propylene glycol;

(B) dissolving the metronidazole in the solvent medium M thus obtained, this dissolution being optionally followed by dilution of the medium obtained by addition of water, whereby a solution S of metronidazole is obtained; and then

(C) mixing the solution S obtained with a gelling polymer, in a sufficient quantity to ensure gelling of the composition.

For the purposes of the present description, the expression “metronidazole-based aqueous gel” means a gelled aqueous phase, preferably monophasic, and containing metronidazole. Advantageously, this gel contains metronidazole as the sole active ingredient eliciting a cosmetic or pharmaceutical effect.

### DETAILED DESCRIPTION OF BEST MODE AND SPECIFIC/PREFERRED EMBODIMENTS OF THE INVENTION

Preferably, the metronidazole is present in the gel in a therapeutically effective amount. This metronidazole content may vary according to the application envisaged for the gel, but it advantageously ranges from 0.25% to 1.0% by mass, and it is typically on the order of 0.75% to 0.8% by mass relative to the total mass of the aqueous gel.

According to the present invention, it has now been demonstrated that, during the preparation of such an aqueous gel, a dissolution of the metronidazole in a medium initially containing water and propylene glycol, of the M medium type in step (B), makes it possible to obtain aqueous metronidazole gels having high stability. Unexpectedly, the present invention has in particular made it possible to establish that the use of this specific step of dissolution in a propylene glycol-based medium makes it possible to obtain gels with increased stability compared to the gels obtained according to the methods of U.S. Pat. No. 4,837,378, where the metronidazole is first dissolved in water, and where propylene glycol is introduced into the medium afterwards.

Even more surprisingly, this stabilization effect is found to be most particularly pronounced when the propylene glycol/water ratio by mass in the medium M formed in step (A) ranges from 2% to 15%, typically from 3% and 10%. Thus, the method of the invention requires in general much less propylene glycol than the method of U.S. Pat. No. 4,837,378, where this compound is introduced in an amount of 7.9% by mass (weight) into the metronidazole dispersion. This addition of propylene glycol in a limited quantity constitutes another advantage of the method of the invention, in particular from an economic point of view.

Without wishing to be bound by any particular theory, it is possible that the stabilizing effect according to the invention is at least partly due to the fact that a mixture of water and propylene glycol allows a very good solubilization of metronidazole, in particular when the water and the propylene glycol are present in the abovementioned advantageous ranges, whereas a lower solubilization is obtained when the propylene glycol is introduced after the dissolution of the metronidazole in water, even when the propylene glycol is post-introduced in large quantities as in the method of U.S. Pat. No. 4,837,378. In this regard, it has now been demonstrated that while metronidazole has a solubility in water of the order of 0.78%, water/propylene glycol mixtures can effect higher solubilities.

According to an advantageous embodiment of the method of the invention, medium M formed in step (A) comprises a preservative in addition to the water and the propylene glycol, this preservative being preferably present in an amount effective to inhibit microbial growth in the gel during its storage. Advantageously, this preservative is selected from the paraben family. In particular, a mixture of methylparaben and of propylparaben is found to be particularly advantageous to this effect. In particular, in these amounts, the presence of preservatives such as parabens in medium M also makes it possible, in certain instances, to improve the solubilization of metronidazole during step (B), and it also leads to an improvement in the stability of the gel obtained.

Medium M most often is a mixture of water, propylene glycol and optionally preservatives of the abovementioned type, excluding any other compound. Nevertheless, the presence of other chemical species is not excluded insofar as they do not hamper effective solubilization of metronidazole in step (B).

Thus, according to another particular embodiment, the medium of step (A) may for example comprise ethylene diamine tetraacetic acid (EDTA) or one of its salts as additional ingredient, although it is most often preferable for this acid to be introduced after the dissolution of metronidazole in step (B).

EDTA, which is commonly used in dermatological compositions, is useful in particular for chelating metal cations which may be present as impurities in the composition, which makes it possible in particular to avoid undesirable side effects in certain patients. EDTA also inhibits the phenomena of browning of the composition which can occur during storage of the gel, in particular when the pH of the gel is on the order of 3.5 to 5.4. The gel prepared according to the method of the present invention advantageously contains EDTA, preferably in an amount of 0.01% to 0.1% by mass, and typically in an amount on the order of 0.05% by mass.

Regardless of the exact composition of the medium M formed in step (A), it is preferable, in general, that, in step (B), the dissolution of metronidazole in this medium M is carried out at a temperature greater than 40° C., this temperature being advantageously at least 45° C., and most preferably at least 50° C., in particular in order to obtain optimum dissolution of the metronidazole. It is preferable, moreover, that this temperature remains less than 70° C., and preferably less than or equal to 60° C., in particular for economic reasons. Thus, this temperature is typically on the order of 50° C. to 55° C.

Moreover, in step (B), the metronidazole is introduced into medium M with a mass ratio (metronidazole/medium M) which is most often from 0.5 to 2.5, for example from 0.8 to 2.2, typically from 1 to 2.

According to a particular embodiment, step (B) simply entails dissolving metronidazole in medium M prepared in step (A).

Another embodiment of step (B) entails dissolving, in a first instance, the metronidazole in medium M prepared in step (A), and then in diluting the metronidazole solution by adding water. According to this embodiment, the water is in general introduced alone as dilution medium, but it is not impossible, according to another particular embodiment, for the water for dilution to be introduced in the form of an aqueous solution containing water-soluble compounds, for example EDTA.

Regardless of the embodiment of step (B), this step leads to a solution S of metronidazole in aqueous medium being obtained. This solution S has, in general, a metronidazole concentration greater than 0.5% by mass, and it is typically from 0.7% to 2.5% by mass, relative to the total mass of solution S. In this solution, the metronidazole is mainly in solubilized form. Thus, in general, in solution S obtained at the end of step (B), less than 1%, and most often less than 0.5%, or even less than 0.1%, of the total mass of metronidazole is in the insolubilized state.

Step (C) of mixing solution S with the gelling polymer may be carried out by any means known to one skilled in the art. Thus, the gelling agent may in particular be simply introduced into solution S. Alternatively, the polymer may first be mixed with water, and optionally with other water-soluble compounds such as EDTA or pH-regulating agents such as sodium hydroxide, to form a pre-gel, this pre-gel then being mixed with solution S.

The gelling polymer employed in step (C) may be any polymer suitable for gelling an aqueous phase in order to form a gel of uniform structure. This is preferably a water-dispersible polymer, having high affinity for water, this polymer being preferably a polymer having free carboxyl groups which are completely or partly neutralizable in the form of carboxylates with a base. Particularly advantageous gelling polymers in step (C) are vinyl polymers with water-dispersible carboxylate groups, in particular poly(acrylic acids) neutralized with a base. Most particularly advantageous are polymers having a molecular mass on the order of 1,250,000 to 4,000,000. Thus, suitable poly(acrylic acids) are for example the poly(acrylic acids) crosslinked with compounds of the polyalkenyl polyether type, such as carbomers, such as the polymers available from the company Goodrich under the trademarks Carbopol 934, 940 or 941, Carbopol 940 being most particularly preferred.

The neutralization of the carboxyl groups in the gelling polymers described above may be carried out by adding to the gel a base, for example aqueous ammonia, NaOH or organic amines, such as alkylamines (for example methylamine, ethylamine) or alternatively dialkylamines, trialkylamines, alkanolamines or dialkanolamines. In this context, it should be noted that the metronidazole itself is sufficiently basic to bring about partial neutralization of the acid groups and thus bring about at least an onset of gelling. The final pH of the gel may vary widely according to the application envisaged. However, most often, this pH is on the order of 3 to 6.9, and preferably from 4 to 5.5.

The gelling polymer is introduced in step (C) in a sufficient quantity to lead in fine to a gel being obtained. Advantageously, this quantity is sufficient to produce a viscosity suitable for topical application of the gel, in particular for dermatological applications. In particular, to obtain such a viscosity, the gelling polymer is advantageously introduced in step (C) in a quantity such that its final content in the gel is from 0.2% to 7.0% by mass, preferably from 0.5% to 1.5% by mass, relative to the total mass of the gel, this content being typically on the order of 0.6% by mass.

According to another advantageous embodiment, steps (A) to (B) of the method of the invention entail:

(A1) forming a medium M comprising water and propylene glycol, preferably in combination with preservatives and EDTA;

(B1) dissolving the metronidazole in the aqueous medium M thus formed, so as to form a solution S of metronidazole; and then

(C1) introducing into the solution S obtained a gelling polymer in a sufficient quantity to bring about gelling of the composition.

According to this embodiment, steps (A1) to (C1) advantageously comprise:

(a1) forming a medium M comprising from 96% to 98% of water, from 2% to 5% (preferably from 2.5% to 3.5%) of propylene glycol, from 0% to 2% (preferably from 0.5% to 1.5%) of preservatives (typically a mixture of methylparaben and propylparaben) and from 0% to 2% (preferably on the order of 0.8% to 1.2%, and typically on the order of 1%) of EDTA;

(b1) dissolving metronidazole in the aqueous medium M thus formed, with a metronidazole/medium M ratio by mass of from 0.5% to 1% (advantageously on the order of 0.7% to 0.9%) so as to form a solution S of metronidazole; and then

(c1) introducing into the resulting solution S a gelling polymer carrying free carboxyl groups neutralized in the from of carboxylates, with a gelling polymer/solution S ratio by mass of from 0.5% to 1%, typically from 0.6% to 0.8%.

According to another advantageous embodiment, steps (A2) to (C2) of the method of the invention entail:

(A2) forming a medium M comprising water and propylene glycol, preferably in combination with preservatives;

(B2) dissolving the metronidazole in the aqueous medium M1 thus formed, so as to form a solution S′ of metronidazole, and then adding water to this solution S′, whereby a solution S of metronidazole is obtained; and then

(C2) mixing the resulting solution S with a gelled aqueous phase containing a gelling polymer, in a sufficient quantity to bring about gelling of the entire composition, preferably in combination with EDTA.

According to another advantageous embodiment, these steps (A2) to (C2) entail:

(a2) forming a medium M comprising 90% to 95% of water, 5% to 10% (preferably from 7% to 8%) of propylene glycol, and from 0% to 0.5% (preferably from 0.2% to 0.4%) of preservatives (preferably a mixture of methylparaben and propylparaben);

(b2) dissolving metronidazole in the aqueous medium M thus formed, with a metronidazole/medium M ratio by mass of from 1.5% to 2.5% (advantageously on the order of 1.9% to 2.2%) so as to form a solution S′ of metronidazole, and then adding water to this solution S′ so as to obtain a solution S of metronidazole having a metronidazole content of 1% to 2.2%, preferably from 1.8% to 2.1% by mass (typically on the order of 1.9% by mass); and then

(c2) mixing the resulting solution S with a gelled aqueous phase containing 98% to 99.5% of water, 0.5% to 1.5% (preferably 0.8% to 1.1%) of a gelling polymer carrying free carboxyl groups neutralized in the form of carboxylates, with a gelling aqueous phase/solution S ratio by mass of from 1.4:1 to 1.8:1, typically from 1.5:1 to 1.7:1.

Regardless of its embodiment, the method of the invention makes it possible to obtain metronidazole-based aqueous gels which have a high stability during their storage. These particularly stable compositions constitute a second aspect of the present invention.

The subject of the present invention also relates to compositions, in particular aqueous gels, which are obtained according to one of the methods described above.

These compositions, more particularly these aqueous gels, are in particular suitable for the preparation of a medicament intended for application by the topical route for the prophylactic or therapeutic treatment of skin conditions, in particular in human beings.

According to the present invention, these compositions, in particular these gels, are in particular suitable for the treatment of rosacea, or of various forms of acne, such as acne vulgaris, acne conglobata or nodulocystic acne, or certain types of dermatitis, such as perioral or seborrhoeic dermatitis.

These various applications of the gels which are obtained according to the abovementioned steps (A) to (C) further constitute another aspect of the present invention.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Method for Preparing an Aqueous Gel G1 Based on Metronidazole

An aqueous gel G1 based on metronidazole was prepared under the following conditions:

Preparation of a Solvent Medium Based on Water and Propylene Glycol:

In a manufacturing tank maintained at 50° C. +/−3° C., 900 kg of purified water, 30 kg of propylene glycol and 0.5 kg of EDTA (ethylenediaminetetraacetic acid) were mixed. The mixing was carried out with the aid of a scraper at 25 revolutions per minute and a turbine at 1,000 revolutions per minute, with a mixing period of 10 minutes.

0.8 kg of methylparaben and 0.2 kg of propylparaben were then added to the medium. The medium was then homogenized with the aid of a scraper at 25 revolutions per minute and a turbine at 3,000 revolutions per minute, under a −0.3b vacuum and at a temperature of 50 +/−3° C. for 20 minutes.

A homogeneous solvent medium based on water and propylene glycol was thus obtained.

Dissolution of Metronidazole:

7.5 kg of metronidazole were introduced into the solvent medium thus prepared. The dissolution was carried out while the medium was maintained at 50° C. +/−3° C., with stirring with the aid of a scraper at 25 revolutions per minute and a turbine at 1,000 revolutions per minute, under a −0.3 b vacuum, for 15 minutes.

A solution (s1) of metronidazole at about 0.8% by mass was thus obtained.

Gelling:

The solution (s1) obtained was gelled by adding 6.5 kg of Carbomer 940 (marketed by Goodrich), in a dry form. The gelling agent was introduced gradually, with stirring with the aid of a scraper at 25 revolutions per minute and a turbine at 3,000 revolutions per minute, for 30 minutes.

### EXAMPLE 2

### Method for Preparing an Aqueous Gel G2 Based on Metronidazole

An aqueous gel G2 based on metronidazole was prepared under the following conditions:

Preparation of a Solvent Medium Based on Water and Propylene Glycol:

A mixture of 1.04 kg of methylparaben and 0.26 kg of propylparaben was gradually added to 36 kg of propylene glycol, with stirring. The stirring was maintained until complete dissolution of the methylparaben and propylparaben had been obtained.

The medium thus obtained was introduced into 429 liters of purified water, maintained stirred in a 700 liter jacketed reactor, at a temperature of from 50° C. and 55° C. The mixing was carried out with the aid of a dispersing device.

A homogeneous solvent medium based on water and propylene glycol was thus obtained.

Dissolution of Metronidazole:

9.75 kg of metronidazole were introduced into the solvent medium thus prepared. The dissolution was carried out in the jacketed reactor and with the aid of the dispersing device of the preceding step, while the medium was maintained from 50° C. and 55° C.

A solution of metronidazole (S2) at about 2% by mass was thus obtained.

Gelling:

In a 1500 liter reactor, 772 kg of purified water were mixed with 0.65 kg of EDTA (ethylenediaminetetraacetic acid) and 7.8 kg of Carbomer 940 NF (Goodrich). The mixing was carried out with stirring at 850-1,100 revolutions per minute, for 60 minutes.

A solution of 1.3 kg of NaOH in 13 liters of water was then introduced into the medium. The medium was then subjected to stirring at 30 revolutions per minute for 90 minutes.

At the end of the stirring, an aqueous phase gelled with Carbomer 940 NF was obtained.

This gelled phase was mixed with the solution (s2) of metronidazole obtained above, at 55° C. The mixture was prepared by introducing the solution (s1) into the gelled aqueous phase, allowing the medium to cool to 33° C., and then mixing with coaxial stirring at 30 revolutions per minute for 120 minutes.

At the end of the stirring, a gel G2 based on metronidazole was obtained.

### EXAMPLE 3

### Method for Preparing an Aqueous Gel G3 Based on Metronidazole

An aqueous gel based on metronidazole was prepared under the following conditions:

Preparation of a Solvent Medium Based on Water and Propylene Glycol:

60 kg of propylene glycol, 1.6 kg of methylparaben and 0.4 kg of propylparaben were mixed.

The mixture thus obtained was introduced into 660 liters of purified water, maintained stirred at 1,000 revolutions per minute in a reactor maintained at a temperature of 53° C. +/−2° C.

A homogeneous solvent medium based on water and propylene glycol was thus obtained.

Dissolution of Metronidazole:

15 kg of metronidazole were introduced into the solvent medium thus prepared. The dissolution was carried out while the medium was maintained at 53° C. +/−2° C., with a turbine stirring at 1,000 revolutions per minute, with a coaxial at 30 revolutions per minute, for 5 minutes.

A solution of metronidazole (S3) at about 2% by mass was thus obtained.

Gelling:

In a 2,000 liter reactor, 1148 kg of purified water were mixed with 1 kg of EDTA (ethylenediaminetetraacetic acid), with turbine stirring at 1,000 revolutions per minute, with a coaxial at 30 revolutions per minute, for 5 minutes. 12 kg of Carbomer 940 NF (Goodrich) were then added to this medium under a −0.4 b vacuum. The mixing was carried out with stirring at 1000 revolutions per minute for 60 minutes.

A solution of 2 kg of NaOH in 20 liters of purified water was then introduced into the medium. The medium was then subjected to stirring at 1000 revolutions per minute, under a −0.6 b vacuum, for 90 minutes.

At the end of the stirring, an aqueous phase gelled with the Carbomer 940 NF was obtained.

The solution (s1) of metronidazole prepared above was mixed with this gelled phase. The mixture was prepared by introducing the solution (s3) prepared above into the gelled aqueous phase, allowing the medium to cool to 31° C. +/−2° C., and then mixing, with coaxial stirring at 30 revolutions per minute under a −0.8 b vacuum, for 120 minutes.

At the end of the stirring, a gel G3 based on metronidazole was obtained.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method for preparing a topically applicable, highly stable aqueous gel which comprises metronidazole, comprising the successive steps:
(A) forming a solvent medium M comprising water and propylene glycol;
(B) dissolving the metronidazole in this solvent medium M, such dissolution optionally being followed by dilution of the medium obtained by addition of water, whereby a solution S of metronidazole is obtained; and thence
(C) mixing the solution S obtained with a gelling polymer, in a sufficient amount to ensure gelling of the composition.

2. The method as defined by claim 1, wherein the propylene glycol/water ratio by mass in the medium M formed in step (A) ranges from 2% to 15%.

3. The method as defined by claim 1, wherein the medium M formed in step (A) additionally comprises a preservative.

4. The method as defined by claim 3, wherein the preservative comprises a mixture of methylparaben and propylparaben.

5. The method as defined by claim 1, wherein step (B), the dissolution of metronidazole in the medium M is carried out at a temperature ranging from 40° C. to 60° C.

6. The method as defined by claim 1, wherein step (B), the metronidazole is introduced into the medium M wherein the ratio by mass (metronidazole/medium M) ranges from 0.5% to 2.5%.

7. The method as defined by claim 6, wherein the solution of metronidazole S obtained at the end of step (B) has a metronidazole concentration greater than 0.5% by mass, relative to the total mass of the aqueous solution.

8. The method as defined by claim 1, wherein the solution of metronidazole S obtained at the end of step (B), less than 1% of the total mass of metronidazole is in the insolubilized state.

9. The method as defined by claim 1, comprising the steps:
(a1) forming a medium M comprising from 96% to 98% of water, from 2% to 5% of propylene glycol, from 0% to 2% of preservatives and from 0% to 2% of EDTA;
(b1) dissolving metronidazole in the aqueous medium M thus formed, with a metronidazole/medium M ratio by mass of from 0.5% to 1% to form a solution S of metronidazole; and thence
(c1) introducing into the solution S obtained a gelling polymer carrying free carboxyl groups neutralized in the form of carboxylates, with a gelling polymer/solution S ratio by mass ranging from 0.5% to 1%.

10. The method as defined by claim 1, comprising the steps:
(A2) forming a medium M comprising water and propylene glycol;
(B2) dissolving the metronidazole in the aqueous medium M thus formed, to form a solution S′ of metronidazole, and then adding water to this solution S′, whereby a solution S of metronidazole is obtained; and thence
(C2) mixing the solution S obtained with a gelled aqueous phase containing a gelling polymer, in a sufficient amount to effect gelling of the entire composition.

11. The method as defined by claim 10, comprising the steps:
(a2) forming a medium M comprising 90% to 95% of water, 5% to 10% of propylene glycol, and from 0% to 0.5% of preservatives;
(b2) dissolving metronidazole in the aqueous medium M thus formed, with a metronidazole/medium M ratio by mass of from 1.5% to 2.5% to form a solution S′ of metronidazole, and then adding water to this solution S′ to obtain a solution S of metronidazole having a metronidazole content of 1% to 2.2%; and thence
(c2) mixing the solution S obtained with a gelled aqueous phase containing 98% to 99.5% of water, 0.5% to 1.5% of a gelling polymer carrying free carboxyl groups neutralized in the form of carboxylates, with a gelling aqueous phase/solution S ratio by mass ranging from 1.4:1 and 1.8:1.

12. The topically applicable, highly stable aqueous gel which comprises metronidazole, prepared by the method as defined by claim 1.

13. A topically applicable, highly stable aqueous gel containing metronidazole, which comprises a therapeutically effective amount of said metronidazole dissolved in a solvent medium M comprising propylene glycol and water.

14. A topically applicable, highly stable aqueous gel containing metronidazole, which comprises a therapeutically effective amount of said metronidazole first dissolved in a solvent medium M comprising propylene glycol and water and thence diluted with water to provide a solution S of metronidazole, said solution S being admixed with a gelling polymer to effect gelation thereof.

15. The topically applicable, highly stable aqueous gel containing metronidazole as defined by claim 13, wherein the propylene glycol/water ratio by mass in the medium M ranges from 2% to 15%.

16. The topically applicable, highly stable aqueous gel containing metronidazole as defined by claim 14, wherein the propylene glycol/water ratio by mass in the medium M ranges from 2% to 15%.

17. The topically applicable, highly stable aqueous gel containing metronidazole as defined by claim 13, said solvent medium M further comprising at least one preservative and, optionally, at least one other active chemical species.

18. A regime or regimen for the prophylactic or therapeutic treatment of a skin condition, comprising topically applying onto the afflicted skin area of an individual in need of such treatment, a thus effective amount of the highly stable, aqueous gel containing metronidazole as defined by claim 13.

19. A regime or regimen for the prophylactic or therapeutic treatment of an acne, rosacea and/or a dermatitis, comprising topically applying onto the afflicted skin area of an individual in need of such treatment, a thus effective amount of the highly stable, aqueous gel containing metronidazole as defined by claim 13.
